(19) United States
(12) Patent Application Publication
(10) Pub. No.: US 2023/0075782 A1
(43) Pub. Date: Mar. 9, 2023

(54) PROVIDING ASSISTANCE DURING MEDICAL EMERGENCIES WHILE DRIVING

(71) Applicant: International Business Machines Corporation, Armonk, NY (US)

(72) Inventors: Hau Co, Sharon (CA); Joseph Lam, Markham (CA)

(21) Appl. No.: 17/469,440

(22) Filed: Sep. 8, 2021

(51) Int. Cl. A61B 5/18; A61B 5/00

(52) U.S. Cl. CPC A61B 5/18; A61B 5/4094; A61B 5/7282; A61B 5/7275; A61B 5/746; A61B 5/747; A61B 5/7264

(57) ABSTRACT

An approach for providing assistance to an operator of a vehicle during a medical event and/or driving impairment is disclosed. The approach includes determines driving profile of a driver, monitors the driver during a trip, identifies an occurrence of the medical event associated with the driver. The approach generates an initial action list in responsive the occurrence of the medical event. The approach executes the initial action list and determines whether the driver is cognizant. The approach generates a subsequent action list in responsive to the driver is not cognizant and executes the subsequent action list.

### BACKGROUND

The present invention relates generally to the field of transportation, and more particularly to providing emergency assistance to passengers during travel.

Road traffic crashes rank as the 9th leading cause of death and account for 2.2% of all deaths globally. With nearly 1.25 million people dying in road crashes each year, averaging 3,287 deaths a day, drivers' ability to remain focused, to be aware of their surroundings, and to be in full control of the vehicle being operated is vital to ensure road safety for drivers and pedestrians around the world.

A seizure occurs when there is a sudden, uncontrolled electrical disturbance in the brain. It can cause changes in your behavior, movements or feelings, and in levels of consciousness. In the U.S. alone, over 700,000 licensed drivers have epilepsy. Seizures are unpredictable, and even a small one at the wrong time can lead to an injury or death. A person with a seizure disorder that causes lapses in consciousness may be putting the public at risk from their operation of a motor vehicle. In the U.S., people with epilepsy can drive if their seizures are controlled with medication or other treatment and they meet the licensing requirements in their state.

Self-driving cars, also known as AV (autonomous vehicle), are becoming readily available for the majority of users. These AVs can definitely provide great help in people's lives. For example, for those people who are not comfortable with driving or who has certain disabilities that cannot drive can definitely take advantage of this new technology, the AV alleviates that problem.

### SUMMARY

Aspects of the present invention disclose a computer-implemented method, a computer system and computer program product for providing assistance to an operator of a vehicle during a medical event. The computer implemented method may be implemented by one or more computer processors and may include, determining driving profile of a driver; monitoring the driver during a trip; identifying an occurrence of the medical event associated with the driver; in responsive the occurrence of the medical event, generating an initial action list; executing the initial action list; determining whether the driver is cognizant; in responsive to the driver is not cognizant, generating a subsequent action list; and executing the subsequent action list.

According to another embodiment of the present invention, there is provided a computer system. The computer system comprises a processing unit; and a memory coupled to the processing unit and storing instructions thereon. The instructions, when executed by the processing unit, perform acts of the method according to the embodiment of the present invention.

According to a yet further embodiment of the present invention, there is provided a computer program product being tangibly stored on a non-transient machine-readable medium and comprising machine-executable instructions. The instructions, when executed on a device, cause the device to perform acts of the method according to the embodiment of the present invention.

### BRIEF DESCRIPTION OF THE DRAWINGS

Preferred embodiments of the present invention will now be described, by way of example only, with reference to the following drawings, in which:

FIG. 1 is a functional block diagram illustrating a vehicle assistance environment, designated as 100, in accordance with an embodiment of the present invention;

FIG. 2 is a diagram illustrating the functionality of vehicle component 111 as it relates to a user with an onset of seizure, in accordance with one embodiment of the present invention;

FIG. 3 is a high-level flowchart illustrating the operation of vehicle component 111, designated as 300, in accordance with an embodiment of the present invention; and

FIG. 4 depicts a block diagram, designated as 400, of components of a server computer capable of executing the vehicle component 111 within the vehicle assistance environment, of FIG. 1, in accordance with an embodiment of the present invention.

### DETAILED DESCRIPTION

The current state of art as it pertains for users with medical disabilities or driving impairments driving a vehicle can present some challenges. For example, if the user (i.e., driver) develops a seizure while driving, it could lead to loss of control of the vehicle, property damage and possibility fatalities.

Embodiments of the present invention recognizes the deficiencies in the current state of art as it relates to assisting users (i.e., driver) with medical issues/disabilities during a medical emergency/events (associated with the medical issues) or driving impairments while the user is operating a vehicle and provides an approach for, (i) lessening and/or preventing the impact of a potential loss of control of the vehicle and (ii) assisting the driver. One approach may consist of (i) notifying the other self-driving cars around to avoid the vehicle of the user and/or (ii) activating the necessary functions in the car to bring the car to a safe stop out of traffic. The approach may include, observing and monitoring the user while driving through the use of cameras, IoT devices and other wearable electronic devices (e.g., watch, glucose monitoring, etc.). Embodiment may recognize that an imminent medical emergency (e.g., seizure, epilepsy episode, etc.) may occur to the user and embodiment may take appropriate actions to minimize the effect to the user and/or to the vehicle (in case the user is unable to safely operate the vehicle during the drive). For example, userA has been diagnosed with seizure. The vehicle recognizes userA is prone to seizures through, i) registering that information (with userA's permission) with the vehicle or ii) observation and/or monitoring the user (comparing against a medical database). UserA begins to drive the vehicle from home to work (typically a 30 minute drive). After 10 mins, userA begins to exhibit characteristics that it indicative (precursors) to a seizure event. The vehicle (through monitoring of userA) recognizes a danger where userA will be unable to operate the vehicle and determines the best course of action is to, either (i) steer the vehicle to safety (should the user become incapacitated) or (ii) instruct the user to steer to safety (assuming the user is semi-conscious/aware).

Some embodiments may provide the ability to manage other medical disabilities such as, (i) possible onset of a heart attack or stroke, (ii) extreme intoxication (e.g., alcohol or substance abuse) and iii) other medical disease (e.g., diabetes, epilepsy, cardiovascular, alcoholism and mental illness). The embodiment can monitor implantable devices such as the driver's insulin pump, coronary stent, heart pacemaker, cardioverter defibrillator or sensors. Embodiments will observe levels, treads and patterns and the possible onset of a health issue that could impair driving. For example, embodiments may observe and pre-screen for the possible onset of a heart attack or stroke. In another example, embodiments may observe and pre-screen for severe intoxication.

Some embodiments may provide the ability to connect to the driver's external wearable devices (e.g., smart watch, smart wristbands, etc.) which could contain health information stored in health-related application, such as, HealthTap®, Moodpath® and Weight Watcher® to observe or pre-screen for possible onset of driving impairment.

Some embodiments can provide for a complete and expandable system that proactively, and with a fair degree of confidence, detects the onset of different medical conditions, and in response to this, execute preventive measures to mitigate or at least minimize the impact of fatal car accidents.

Some embodiments can provide more preventive measures to eliminate or lessen the impact of a car accident, including, but is not limited to, slowing down of the vehicle to safety, relegating the control of the vehicle to the vehicle computer, pulling the vehicle to the side of the road or into a parking lot, audio prompts, tightening of the seatbelt, contacting emergency contacts, and transporting the driver to the nearest clinic/hospital.

Some embodiments can also outline where key events are recorded in-camera to provide contextual information that might be useful to the medical staff at clinic/hospital, insurance company, and legal entities should an accident occur.

Some embodiments can also prescreen drivers who are more likely to develop a seizure as a result of genetics, prior head injuries that increase the likely of developing seizures, post-operations, changes in health such as having a stroke, or an infection such as meningitis or another illness.

Some embodiments may instruct the surrounding vehicles to avoid being hit by the user's car. For example, embodiment may instruct passing autonomous vehicles to perform blockade with warning lights enabled. Thus, this can provide greater access for emergency services/responders.

Some embodiments can check the user's vital signs and use image recognition technology to determine whether the rider has suffered injuries or not. If the user has injuries, then system may determine the extent of the injuries based on sensors (i.e., biosensors).

Some embodiments may recognize non-medical conditions (or driving impairments) that can affect the driver from operating the vehicle safely and effectively. For example, driving impairments can include, but it is not limited to, drunkenness and substance abuse.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments, whether or not explicitly described.

The terms “user”, “rider” and “driver” may be used interchangeably throughout the disclosure but will have similar meaning (i.e., someone operating a vehicle).

It should be understood that the Figures are merely schematic and are not drawn to scale. It should also be understood that the same reference numerals are used throughout the Figures to indicate the same or similar parts.

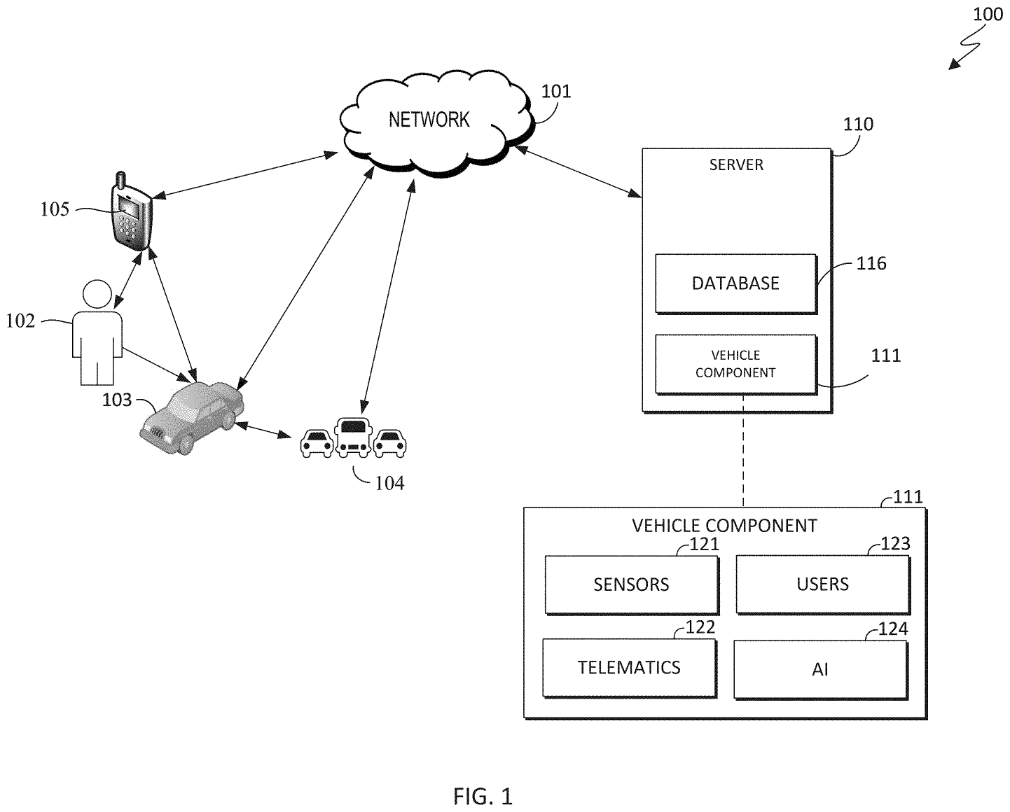

FIG. 1 is a functional block diagram illustrating a vehicle assistance environment, designated as 100, in accordance with an embodiment of the present invention. FIG. 1 provides only an illustration of one implementation and does not imply any limitations with regard to the environments in which different embodiments may be implemented. Many modifications to the depicted environment may be made by those skilled in the art without departing from the scope of the invention as recited by the claims.

Vehicle assistance environment 100 includes network 101, users 102, user vehicle 103, other vehicles 104, devices 105 and server 110.

Network 101 can be, for example, a telecommunications network, a local area network (LAN), a wide area network (WAN), such as the Internet, or a combination of the three, and can include wired, wireless, or fiber optic connections. Network 101 can include one or more wired and/or wireless networks that are capable of receiving and transmitting data, voice, and/or video signals, including multimedia signals that include voice, data, and video information. In general, network 101 can be any combination of connections and protocols that can support communications between server 110 and other computing devices (not shown) within Vehicle assistance environment 100. It is noted that other computing devices can include, but is not limited to, any electromechanical devices capable of carrying out a series of computing instructions.

Users 102 are drivers and/or passengers of a vehicle. Users may have certain physical and/or mental disabilities and medical issues. Some physical disabilities can include visual and auditory impairment. Mental disabilities may include learning disabilities, etc. Medical issues may include, but it is not limited to, seizures, diabetes, epilepsy, intoxication and heart condition.

User vehicle 103 are vehicle (e.g., autonomous or semi-autonomous) belonging and/or being utilized by the user (i.e., users 102). User vehicle 103 may be full self-driving vehicle (i.e., level 5) or partial driving capability (i.e., levels 1-4) as defined by the SAE (society of automotive engineers).

User vehicle 103 may also contain myriad of onboard sensors (e.g., IoT devices, heart rate, microphone, etc.) that can detect vehicle telemetry and determine user cognitive state/mood. Additionally, a mobile device can act in similar manners to determine the user/driver's cognitive state/mood.

Furthermore, user vehicle 103 can be equipped with safety features, such as, but it is not limited to, ability to lock the seatbelt of the driver, ability to prepare side airbags to deploy (if equipped), ability to prepare the steering wheel airbags to deploy, and any standard safety features associated with vehicle (e.g., anti-lock brakes, etc.).

It is noted that for a semi-autonomous or non-autonomous vehicle, it is possible to retrofit a hardware interface to the car's ECU via OBDII port or other vehicle connection. The hardware interface would communicate directly with a mobile device which can be mounted on the vehicle (assuming the vehicle is not equipped with IoT sensors and other onboard sensors). A mobile device can act in similar manners to determine the user/driver's cognitive state/mood and can be capable of running vehicle component 111.

Other vehicles 104 are vehicles (e.g., non-autonomous or fully autonomous vehicles) that are in the vicinity of user vehicle 103. These vehicles maybe potentially involved in an accident with user.

Devices 105 are smart devices that belongs to the passengers/users. Generally, devices 105 are electronic devices that can interface with the users to provide assistance with everyday activities. In an example, devices 105 can implantable devices such as a heart pacemaker and glucose monitoring system. In another example, devices 105 can be an auditory implant that allows a hearing impaired person to “hear” sounds. Alternatively, devices 105 can be a portable braille device that allows visually impaired users to “read” and it is coupled to an auditory earphones that provides walking navigation instructions. In other examples, devices 105 can be a smart phone, smart watch that can measure heart rate and has other biometric functionality and smart earbuds.

Server 110 can be a standalone computing device, a management server, a web server, a mobile computing device, or any other electronic device or computing system capable of receiving, sending, and processing data. In other embodiments, server 110 can represent a server computing system utilizing multiple computers as a server system, such as in a cloud computing environment. In another embodiment, server 110 can be a laptop computer, a tablet computer, a netbook computer, a personal computer (PC), a desktop computer, a personal digital assistant (PDA), a smart phone, or any other programmable electronic device capable of communicating other computing devices (not shown) within vehicle assistance environment 100 via network 101. In another embodiment, server 110 represents a computing system utilizing clustered computers and components (e.g., database server computers, application server computers, etc.) that act as a single pool of seamless resources when accessed within vehicle assistance environment 100.

Embodiment of the present invention can reside on server 110, integrated with the vehicle or on mobile devices of the user. Server 110 includes vehicle component 111 and database 116.

Vehicle component 111 provides the capability of assisting driver (i.e., users 102) during a medical incident. At a high level of one embodiment, the steps of includes, (i) observing and monitoring for the possible onset of a medical incident (e.g., seizure, etc.) or driving impairments, (ii) executes in-vehicle safety measure for the user, (iii) in-vehicle surveillance begins to document events and/or (iv) vehicle transports driver and passengers to near-by hospital. A medical incident/event is a medical event that is associated with a medical issue. Medical issues may include, but it is not limited to, seizures, diabetes, epilepsy, intoxication and heart condition. For example, a medical event/incident related to someone with a known heart condition is a heart attack. Another example, a medical event/incident related to someone with known epilepsy is a seizure. There are more samples of symptoms along with possible corresponding medical condition in Tables 1 through 3. It is noted that the examples provided for medical event/incident and/or medical issues is not an exhaustive list. Tables 4 through Table 5 illustrates other non-medical issues (driving impairments) that may affect the driver.

Although, medical events are signs/symptoms for a certain medical condition, it is not a substitute for a proper medical diagnosis by trained professionals. Similarly, driving impairments, such as, drunkenness, substance abuse and/or emotional conditions, as illustrated by Tables 4 and Table 5, are signs/symptoms of impairment that can affect the driver but is it not a substitute for a proper diagnosis by trained professionals. Embodiment is not a medical device but as having the corpus of knowing if the driver has, a medical condition/device helps the system to determine the likelihood of a medical event.

In other embodiments, other steps of vehicle component 111 may include, (i) alerting/notifying other vehicles (i.e., other vehicles 104) of the medical incident suffered by the user and/or (ii) alert/notify first responders with the location of the vehicle, status of user (including any information related to the medical issues/disabilities that the user may have) and status of the vehicle (i.e., vehicle is not operable).

Regarding assisting users during/after an accident, vehicle component 111 may leverage a machine learning methodology to determine the best course of action. Any machine learning method for calculating and creating an action list may be used, such as, a Bayesian regression, neural network regression and decision forest regression. For example, vehicle component 111 may analyze the situation before/during/after a medical incident and determine the best course of action for the users by generating an action list. Based on the initially generated action list, vehicle component 111 may recalculate the action list based on changing conditions (e.g., medical symptoms of the user seems more severe than initially observed, delay due to traffic, etc.). Vehicle component 111 will select the best solution from the action list based on the best outcome. For example, the action list may include the following, i) drive users to the nearest hospital (if the vehicle is equipped with full service driving capability), ii) notifying emergency contacts of the user, iii) notify first responders/ambulance (i.e., car is does not have self-driving capability) and iii) alert other vehicles to avoid the scene. Vehicle component 111 and subcomponents will be described in greater details in a subsequent section. It is noted that the action list is not exhaustive.

Vehicle component 111 contains subcomponents (see FIG. 1): sensors component 121, telematics component 122, users component 123 and AI component 124.

As is further described herein below, sensors component 121 of the present invention provides the capability of receiving information from various sensors throughout the vehicle (i.e., users vehicle 103) and receiving/sending information from devices 105 (i.e., belonging to the user). These sensors are used for the following, but it is not limited to, determine the mental and/or physical state of the users, detect other nearby vehicles and detecting hand pressure on the steering wheel of the vehicle.

As is further described herein below, telematics component 122 of the present invention provides the capability of monitoring the vehicle of the users and other surrounding/nearby vehicles. Telematics component 122 can determine if the vehicle of the users is operable (before and/or after an accident) based on system diagnostic of batteries, motors, door sensors, wheels, drivetrain, etc. Telematics component 122 can determine other vehicles nearby based on GPS signal (from other user's mobile device or onboard vehicle communication system).

In some embodiments, telematics component 122 may have the capability to manage operations of a vehicle by interfacing with an ECU (electronic control unit). The operations may include, but it is not limited to, slowing driving (i.e., braking), steering (if the vehicle is capable) and activating onboard safety systems.

As is further described herein below, user component 123 of the present invention provides the capability determining the profile of users (e.g., passengers, drivers, etc.) after they enter vehicle (e.g., via smartwatch, object recognition, etc.). After recognizing the user has entered the vehicle, users component 123 can query database 116 to determine if the users have a medical disability/issues and/or driving impairments (e.g., drunkenness, emotional distress, etc.) and what type of medical issues/disability (assuming the users have given permission to disclose such information and full names may not be required). This would be an opt-in profile based description that the user/passenger would allow to be shared with the vehicle without an invasion of their personal privacy or personal health information. For example, the vehicle would simply know that the user might have visual impairment and suffer from anxiety during a high stress situation.

Some embodiments of users component 123 can perform a baseline driving profile for at the first drive for a new driver of that vehicle. The baseline driving profile can include, speed, lane change, hand pressure (or lack) on the steering wheel, eyes pattern and head and body movement during driving. The baseline driving profile can be used to compare against current drive trip in order to determine if the driver has suffered or is about have a medical event during the drive.

A few examples of medical events, such as, heart attack, stroke, seizure and corresponding possible hardware requirement(s) is listed in the following tables, Table 1, Table 2, and Table 3, respectively.

Table 1 illustrates symptoms, observation made by embodiment and any included hardware requirement for the observation associated with a heart attack. It is noted that the symptoms listed is not an exhaustive list and should not be used as a complete medical diagnosis for various heart conditions.

TABLE 1

| Symptoms (Heart Attack) | System observation | Additional requirement(s) |
|---|---|---|
| Chest pain | One hand on steering wheel | In-vehicle camera |
| | Hand on chest for more than | Pressure sensor on steering |
| | X secs | wheel |
| | Repeated hand movement on | |
| | chest | |
| Pain or discomfort in the | One hand on steering wheel | In-vehicle camera |
| jaw or neck | Hand on jaw/neck for more than | Pressure sensor on steering |
| | X secs | wheel |
| | Repeated hand movement | |
| | on jaw or neck | |
| Pain or discomfort on the back | Repeated movement of shoulder | In-vehicle camera |
| | or neck | |
| | Fidgeting which will likely be | |
| | different from baseline profile | |

Table 2 illustrates symptoms, observation made by embodiment and any included hardware requirement for the observation associated with a stroke. It is noted that the symptoms listed is not an exhaustive list and should not be used as a complete medical diagnosis for stroke conditions.

TABLE 2

| Symptoms (Stroke) | System observation | Additional requirement(s) |
|---|---|---|
| Trouble speaking or difficulty | No response from driver after | Microphone |
| understanding speech | audio prompt from vehicle | |
| | Inability to decipher | |
| | driver's verbal response | |
| | Speech does not match baseline | |
| | profile | |
| Numbness or weakness in the | If numbness weakness occurs on | Pressure sensor on steering |
| face, arm, or leg, especially on | right side of body: | wheel |
| one side of the body | Decreased in usage on the right | |
| | hand side of the steering wheel | |
| | Decreased in usage on the right | |
| | foot on the brake/gas paddles | |
| Trouble seeing in one or both | Repeated blinking | In-vehicle camera |
| eyes | Repeated closing of eyes | |
| | Repeating shaking of head | |

Table 3 illustrates symptoms, observation made by embodiment and any included hardware requirement for the observation associated with substance seizures. It is noted that the symptoms listed is not an exhaustive list and should not be used as a complete medical diagnosis for seizures.

TABLE 3

| Symptoms (Seizure) | System observation | Additional requirement(s) |
|---|---|---|
| Staring spell | Lack of eye movement | Pressure sensor on steering |
| | | wheel |
| Temporary confusion | Too much eye movement | Smart Watch paired to car |
| | Lack of focus on key elements | |
| | on the road | |
| Uncontrollable jerking | Lack of at least one hand on | Pressure sensor on steering |
| movements of the arms and legs | steering wheel for 2 secs or | wheel |
| | more | |
| | Lack of consistent hand pressure | |
| | on steering wheel | |
| | Lack of steady foot pressure on | |
| | gas pedal | |
| Loss of consciousness or | Closed eyes | In-vehicle camera |
| awareness | Inappropriate driving posture | Pressure sensor on steering |
| | Hand(s) not on steering wheel | wheel |
| | Car speed not equal to | |
| | surrounding vehicles | |

In some embodiments, other issues may affect/impaired a driver that may not qualify under medical conditions and medical events/issues. These issues may nevertheless still impact the driver from properly operating the vehicle in a safe and effective manner. These issues are illustrated in Table 4 and Table 5.

Table 4 illustrates symptoms, observation made by embodiment and any included hardware requirement for the observation associated with a drunkenness. It is noted that the symptoms listed is not an exhaustive list and should not be used as a complete medical diagnosis for drunkenness.

TABLE 4

| Symptoms | | |
|---|---|---|
| (Drunkenness) | System observation | Additional requirement(s) |
| Elevated spirit | If music is on: | No additional |
| | Sudden, repeating movement, | requirement(s) needed |
| | and swerving with music | |
| Slurred speech | Unable to decipher | Microphone |
| | driver's verbal response | |
| | Volume, intonation and level of | |
| | speech are different from | |
| | baseline profile | |
| Sleepiness | Closing of eyes | In-vehicle camera |
| | Lowering of head position | Pressure sensor on |
| | Decrease in hand(s) pressure | steering wheel |
| | on the steering wheel | |
| | Vehicle slowing or drifting out | |
| | of lane | |
| | Sudden increase/decrease on | |
| | brake/gas pads | |
| Erratic behavior | Surrounding cars honking | Microphone |

Table 5 illustrates symptoms, observation made by embodiment and any included hardware requirement for the observation associated with substance abuse impairment and/or emotion impairment (e.g., anxiety, stress, etc.). It is noted that the symptoms listed is not an exhaustive list and should not be used as a complete medical diagnosis for substance abuse.

TABLE 5

| Symptoms (Substance | | |
|---|---|---|
| abuse/emotional impairment) | System observation | Additional requirement(s) |
| Shaking hands | Noticeable taps or vibrations at | Pressure sensor on steering |
| | the steering wheel | wheel |
| Irregular heartbeat | Irregular heartbeat | Smart Watch paired to car |
| Runny nose; hacking cough | Sounds of coughs or sneezes | Microphone |
| Frequent rubbing of the nose | Driver rubbing nose with hands | In-vehicle camera |
| | or fingers | |
| Frequent twisting of the jaw, | Frequent twisting of the jaw, | In-vehicle camera |
| back and forth | back and forth | |
| Deep breaths | Driver breathing deeply or | Microphone |
| | laboriously | |

Other embodiment of users component 123 can allow users to register their medical disabilities/issues with the vehicle (i.e., information is locally stored on the vehicle) instead of the vehicle querying the information from a network source (e.g., medical profile, medical database, social media, etc.).

As is further described herein below, AI (artificial intelligence) component 124 of the present invention provides the capability of managing the safety of the users (i.e., users 102) before/during/after a medical incident/situations or driving impairments based on, but it is not limited to, data from users component 123, sensors component 121 and telematics component 122.

A use case example of AI component 124 can include observation for the possible onset of a seizure by looking for symptoms such as temporary confusion, a staring spell, an uncontrollable jerking movements of arms and legs, and/or a loss of consciousness or awareness using camera and IoT technologies, and carries out a number of preventative measures help prevent and lessen the impact of a car accident during the seizure.

In the same embodiment, the functionality of AI component 124 may include generating an action list (either initial or subsequent action list) by leveraging machine learning. Any machine learning method for calculating and creating an action list may be used, such as, a Bayesian regression, neural network regression and decision forest regression. The action list can include, but it is not limited to, alerting the driver to be attentive, alerting other nearby vehicles based on the medical condition of the driver, steering the vehicle to safety and/or to the nearest medical treatment facility, opening the trunk/hood of the vehicle to indicate to others that the vehicle and/or driver has an emergency situation, alerting medical personnel at the nearest facility, slowing down the vehicle and activating safety devices in the vehicle. The action list is based on risk factors. These risk factors can include, but it is not limited to, weather, time of drive, traffic, speed of user's vehicle, status/type of vehicle (e.g., is it a full or semi-autonomous vehicle, what level of self-driving capability, etc.), status of the drivers/passengers after the medical incident, and status of nearby first responders. It is noted that the risk factors is not an exhaustive list.

Some embodiments send the rider's condition to the emergency medical responder's organization to get help if the rider is injured. Embodiments continue monitoring the rider's condition to ensure the rider is safe. Embodiments determine whether the rider needs to exit the car once the car stops in a safe area if it is not safe for the rider to continue staying in the car. If the rider is able to exit the car then the embodiments will open the door to let the rider out if he is capable. In the case the rider is injured, embodiments may keep the door closed until the emergency medical services arrive.

Database 116 is a repository for data used by vehicle component 111. Database 116 can be implemented with any type of storage device capable of storing data and configuration files that can be accessed and utilized by server 110, such as a database server, a hard disk drive, or a flash memory. Database 116 uses one or more of a plurality of techniques known in the art to store a plurality of information. In the depicted embodiment, database 116 resides on server 110. In another embodiment, database 116 may reside elsewhere within vehicle assistance environment 100, provided that vehicle component 111 has access to database 116. Database 116 may store information associated with, but is not limited to, specifications of vehicles utilized by the user, profile of the users (including disabilities/medical conditions), symptoms of substance abuse, symptom of emotional distress/condition, road conditions, contact information for first responder, emergency contact information associated with the user, weather conditions, traffic patterns and specifications of other vehicles nearby scene of the accident.

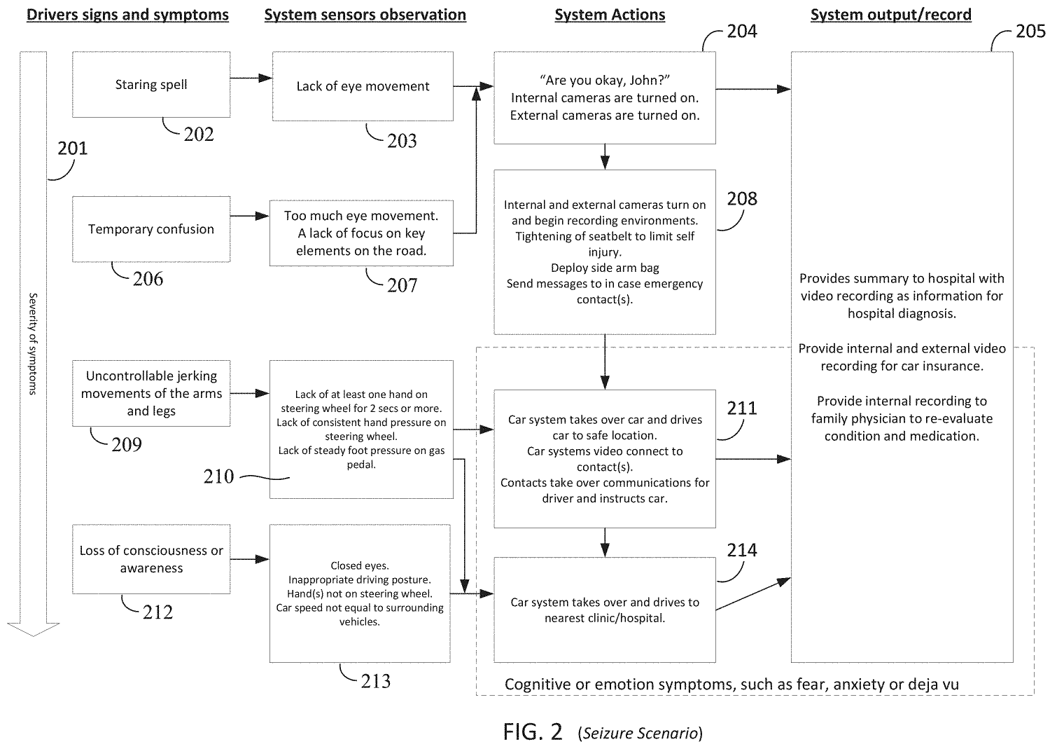

FIG. 2 is a diagram illustrating the functionality of vehicle component 111 as it relates to a user with an onset of seizure, in accordance with one embodiment of the present invention. The diagram illustrates the signs and symptoms of the seizure for a driver. Arrow 201 indicates the severity of the symptoms associated with seizures, where the top is the least and towards the bottom of the Figure as being most severe. The various header blocks (Driver's signs and symptoms, System sensors observation, System Actions and System output/record) indicate the steps (by way of directional arrows) as a symptom occurs. For example, staring spell (block 202) is the least severe signs and symptoms of a seizure (i.e., medical event). Here, embodiments can determine the staring spell by (block 203) by observing the lack of eye movement via IoT devices/sensors. Possible actions by the embodiment (block 204) can include engaging in a dialogue with the driver and turning on various cameras (e.g., internal and external) to record. Information recorded can be used by medical responders and/or insurance companies (block 205).

The next level of severity (from block 202) associated with seizure is block 206. Block 206 denotes “temporary confusion” which can be determined based on following observations of the user (block 207), (i) too much eye movement and (ii) a lack of focus on key elements on the road. The corresponding solutions from the action list (block 208) includes the following, (i) internal and external cameras turn on and begin recording environments, (ii) tightening of seatbelt to limit self-injury, (iii) deploy side arm bag and (iv) send messages to in case emergency contact(s). Information recorded can be used by medical responders and/or insurance companies (block 205).

The next level of severity (from block 206) associated with seizure is block 209, which denotes “uncontrollable jerking movements of arms and legs”. The characteristics/symptoms/sign illustrated by block 209 can be determined based on following observations of the user (block 210). This observation can include, (i) lack of at least one hand on steering wheel for 2 secs or more, (ii) lack of consistent hand pressure on steering wheel and (iii) lack of steady foot pressure on gas pedal. The corresponding options from the action list (block 211) includes the following, (i) car system takes over car and drives car to safe location, (ii) car systems video connect to contact(s) and (iii) contacts take over communications for driver and instructs car. Information recorded can be used by medical responders and/or insurance companies (block 205).

The next level of severity (from block 209) associated with seizure is block 212, which denotes “loss of consciousness or awareness”. The characteristics/symptoms/sign illustrated by block 209 can be determined based on following observations of the user (block 213). This observation can include the following, (i) closed eyes, (ii) inappropriate driving posture, (iii) hand(s) not on steering wheel and (iv) car speed not equal to surrounding vehicles. The corresponding options from the action list (block 214) can include, (i) the car system takes over and drives to nearest clinic/hospital. Information recorded can be used by medical responders and/or insurance companies (block 205). Additionally, the recording (as it relates to system output/record) can provide internal recording to family physician to re-evaluate condition and medication.

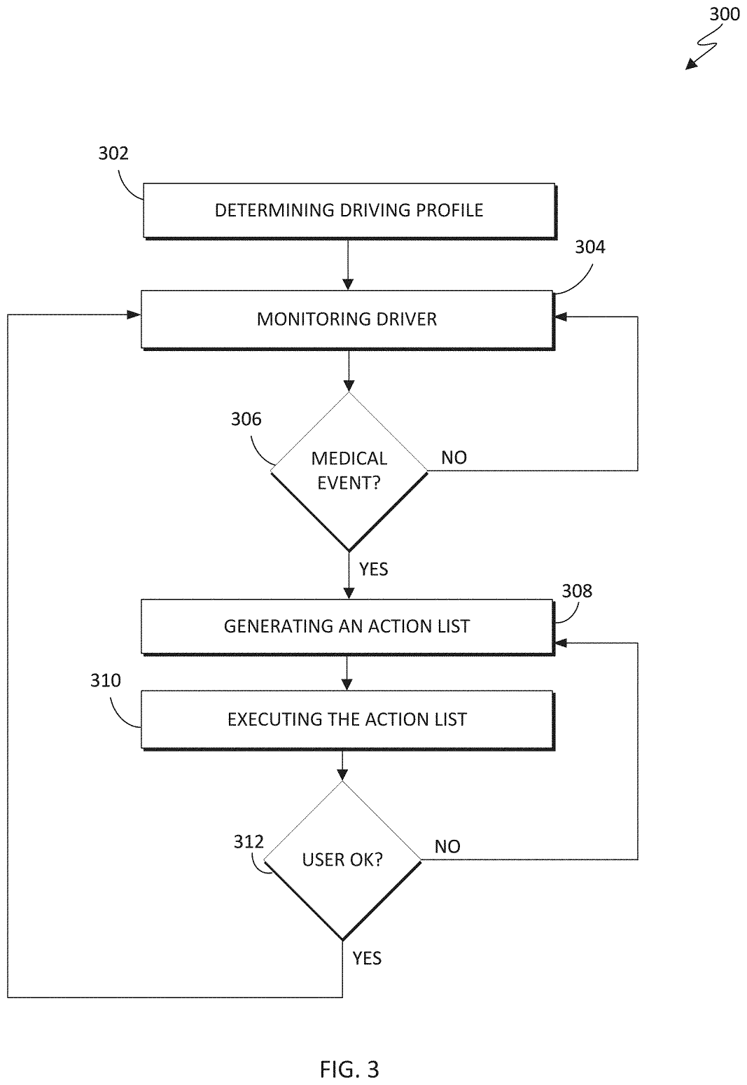

FIG. 3 is a high-level flowchart illustrating the operation of vehicle component 111, designated as process 300, in accordance with one embodiment of the present invention.

Vehicle component 111 determines profile of the driver (step 302). In an embodiment, vehicle component 111, through users component 123 and sensors component 121, determines a baseline driving profile of the driver. The baseline driving profile can include, speed, lane change, hand pressure (or lack) on the steering wheel, eyes pattern and head and body movement during driving. For example, userA is prone to a seizure and drives a semi-autonomous vehicle. Vehicle component 111 has observed userA for a predetermined time in order to create a baseline driving profile. Based on the observation, userA tends to drive with two hands on the steering wheel and keeps a straight eye gaze through the windshield. It is noted that new users (without any driving profile on the vehicle) can be observed while other regular users of the vehicle may already have a driving profile saved.

In some embodiments, vehicle component 111 may determine whether the driver has a medical issues/disabilities as they enter vehicle. In other embodiments, vehicle component 111 may determine that the driver is under the influence of alcohol based on symptoms/signs (see Table 4). For example, userB, has narcolepsy and enters a vehicle to drive to his work. UserB has registered his disability with the vehicle at the initial time (i.e., created a user profile) when the car was purchased. Vehicle recognizes (via camera, sensors, etc.) that userB has enter the car and retrieves the profile of userB.

Vehicle component 111 monitors the driver (step 304). In an embodiment, vehicle component 111, through sensors component 121, continues observing the driving profile/characteristics of the driver during the drive. For example, continuing using userA as an example, vehicle component 111 continuous observing a driving characteristics (i.e., behavior) of userA. The driving behavior can include the following, but it is not limited to, speed, lane change, hand pressure (or lack) on the steering wheel, eyes pattern and head and body movement during driving.

Vehicle component 111 identifies a medical event associated with the driver (decision block 306). In an embodiment, vehicle component 111, through sensors component 121, determines that the driver is exhibiting initial signs/characteristics (e.g., temporary confusion, staring spell, etc.) of an oncoming seizure (“YES” branch of decision block 306). Otherwise, if vehicle component 111 does not detect/determine a medical event (“NO” branch of decision block 306) then vehicle component 111 resumes monitoring the driver (step 304). For example, continuing using userA as an example, vehicle component 111 determines that userA has a staring spell based on the observations signs, such as, eye movement has stopped and starring off into space.

Vehicle component 111 generates an action list (step 308). In an embodiment, vehicle component 111, through AI component 124, creates an action list (e.g., initial and subsequent) corresponding to possible solution. The action list can include, but it is not limited to, alerting the driver to be attentive, alerting other nearby vehicles based on the medical condition of the driver, steering the vehicle to safety and/or to the nearest medical treatment facility, alerting medical personnel at the nearest facility, slowing down the vehicle and activating safety devices in the vehicle. For example, continuing using userA as an example vehicle component 111 creates an action list containing the following: (i) alerting the driver to be more attentive by announcing over the speaker system and the on screen display, (ii) engage in a minor conversation with the driver and (iii) turning on internal and external cameras to record.

In some embodiments, the action list may comprise of, i) either pull the car over for the user if the car is semi/full autonomous (minor, inexpensive modifications can be made to older vehicles, which allows for simple preventative actions such as braking gradually to take place in case of a medical event), ii) alert the driver (or those cars surrounding him) to make them pull over, iii) prep the car for potential crash impact, iv) gradually stopping the vehicle provided that this is safe to do so (vehicle needs to have some level of situation awareness via cameras/sensors), v) attempt to bring the driver back to a state of consciousness (i.e., if appropriate, play a loud sound through the radio), vi.) notify/communicate with emergency contacts of the driver, vii) if appropriate, take over navigation and transport the driver to a nearby clinic or hospital, viii) begin camera recording so there is clear headlight into what transpired during an accident to help with post-accident medical assessment and ix) alert cars surrounding the vehicle of a medical situation so they can avoid the problematic vehicle.

Vehicle component 111 executes the action list (step 310). In an embodiment, vehicle component 111, through AI component 124, selects the solution(s) from the action list and follows through with the selected action(s). For example, continuing using userA as an example, vehicle component 111 selects initial options (ii) and (iii) from the action list, whereby the vehicle engages in a conversation with userA by asking if the userA is ok and turning on the internal and external cameras to record.

Vehicle component 111 determines if the driver is cognizant (decision block 312). In an embodiment, vehicle component 111, through sensors component 121, determines that the driver is not responding to an initial action (“NO” branch of decision block 312) then embodiment proceeds to generate a subsequent action list (i.e., return to step 308). For example, userA is not responding to question from the vehicle (“John, are you ok?”). Vehicle component 111 may generate a subsequent action list, such as, alerting the driver to pull over, preparing the safety devices on the vehicle and preparing the vehicle for a possible emergency stop. It is noted that this may be repetitive step (i.e., generating and executing action list) until the driver is either ok or the vehicle has to stop due to the inactivity of the driver and possibly the need for the driver to seek medical attention. For example, if userA (i.e., John) does not respond that he is ok and begins to exhibit more severity symptoms (e.g., jerking arm movement and legs, etc.) then vehicle component 111 may choose to safely pull the vehicle over on the side off the road until userA has recovered from the seizure (i.e., from the subsequent action list). If userA doesn't recover/regain consciousness in a predetermined amount of time then vehicle component 111 may decide to call first responder and provide all the necessary information (e.g., medical history, location, etc.) for the first responder. It is noted that the action list is not exhaustive.

Otherwise, if the driver responds that he/she is fine (e.g., cognizant, conscious, etc.) then vehicle component 111 (“NO” branch of decision block 312) continues to monitor the driver for the duration of the trip (step 304). It is noted that vehicle component 111 may disengage/stops monitoring as soon as the driver has concluded the trip or has exited the vehicle (after the vehicle has come to a complete stop).

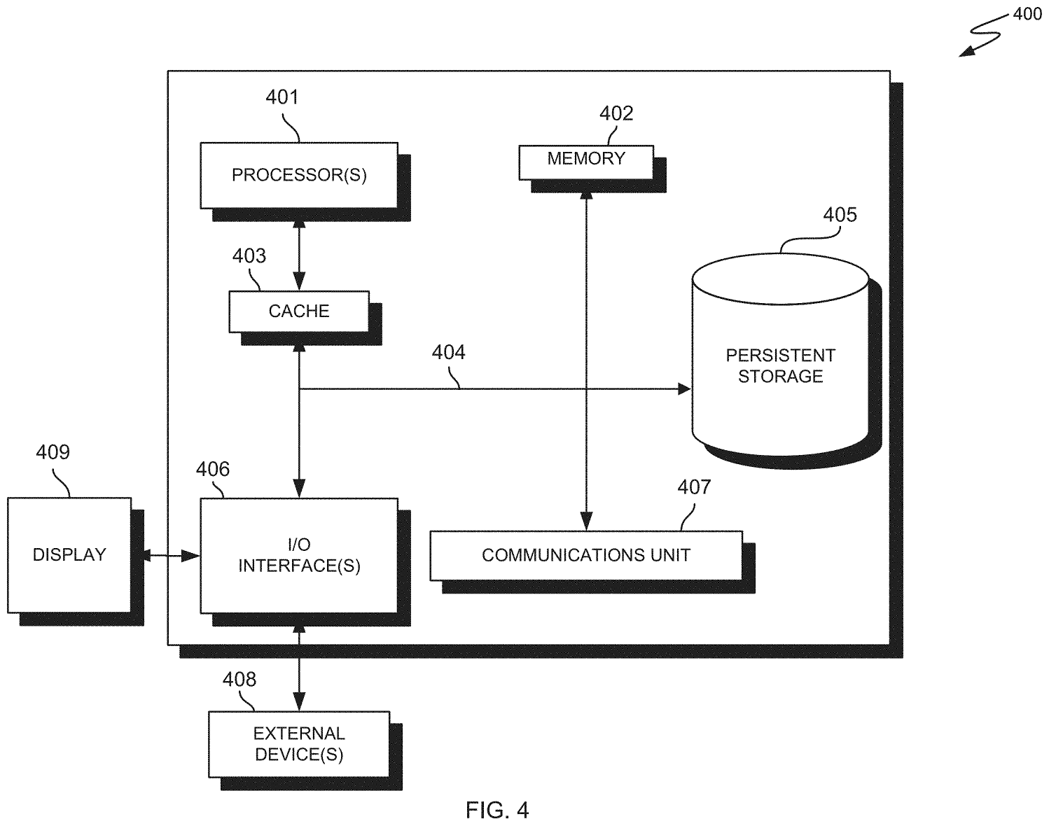

FIG. 4, designated as 400, depicts a block diagram of components of vehicle component 111 application, in accordance with an illustrative embodiment of the present invention. It should be appreciated that FIG. 4 provides only an illustration of one implementation and does not imply any limitations with regard to the environments in which different embodiments may be implemented. Many modifications to the depicted environment may be made.

FIG. 4 includes processor(s) 401, cache 403, memory 402, persistent storage 405, communications unit 407, input/output (I/O) interface(s) 406, and communications fabric 404. Communications fabric 404 provides communications between cache 403, memory 402, persistent storage 405, communications unit 407, and input/output (I/O) interface(s) 406. Communications fabric 404 can be implemented with any architecture designed for passing data and/or control information between processors (such as microprocessors, communications and network processors, etc.), system memory, peripheral devices, and any other hardware components within a system. For example, communications fabric 404 can be implemented with one or more buses or a crossbar switch.

Memory 402 and persistent storage 405 are computer readable storage media. In this embodiment, memory 402 includes random access memory (RAM). In general, memory 402 can include any suitable volatile or non-volatile computer readable storage media. Cache 403 is a fast memory that enhances the performance of processor(s) 401 by holding recently accessed data, and data near recently accessed data, from memory 402.

Program instructions and data (e.g., software and data x10) used to practice embodiments of the present invention may be stored in persistent storage 405 and in memory 402 for execution by one or more of the respective processor(s) 401 via cache 403. In an embodiment, persistent storage 405 includes a magnetic hard disk drive. Alternatively, or in addition to a magnetic hard disk drive, persistent storage 405 can include a solid state hard drive, a semiconductor storage device, a read-only memory (ROM), an erasable programmable read-only memory (EPROM), a flash memory, or any other computer readable storage media that is capable of storing program instructions or digital information.

The media used by persistent storage 405 may also be removable. For example, a removable hard drive may be used for persistent storage 405. Other examples include optical and magnetic disks, thumb drives, and smart cards that are inserted into a drive for transfer onto another computer readable storage medium that is also part of persistent storage 405. vehicle component 111 can be stored in persistent storage 405 for access and/or execution by one or more of the respective processor(s) 401 via cache 403.

Communications unit 407, in these examples, provides for communications with other data processing systems or devices. In these examples, communications unit 407 includes one or more network interface cards. Communications unit 407 may provide communications through the use of either or both physical and wireless communications links. Program instructions and data (e.g., vehicle component 111) used to practice embodiments of the present invention may be downloaded to persistent storage 405 through communications unit 407.

I/O interface(s) 406 allows for input and output of data with other devices that may be connected to each computer system. For example, I/O interface(s) 406 may provide a connection to external device(s) 408, such as a keyboard, a keypad, a touch screen, and/or some other suitable input device. External device(s) 408 can also include portable computer readable storage media, such as, for example, thumb drives, portable optical or magnetic disks, and memory cards. Program instructions and data (e.g., vehicle component 111) used to practice embodiments of the present invention can be stored on such portable computer readable storage media and can be loaded onto persistent storage 405 via I/O interface(s) 406. I/O interface(s) 406 also connect to display 409.

Display 409 provides a mechanism to display data to a user and may be, for example, a computer monitor.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method for providing assistance to an operator of a vehicle during a medical event and/or driving impairment, the computer-method comprising:
determining driving profile of a driver;
monitoring the driver during a trip;
identifying an occurrence of the medical event and/or and driving impairment associated with the driver;
in responsive the occurrence of the medical event, generating an initial action list;
executing the initial action list;
determining whether the driver is cognizant;
in responsive to the driver is not cognizant, generating a subsequent action list; and
executing the subsequent action list.

2. The computer-implemented method of claim 1, wherein determining the driving profile of the driver, further comprises:
generating a baseline driving profile based on a predetermined period of time associated with an observation of the driver; and
storing the baseline driving profile.

3. The computer-implemented method of claim 2, wherein monitoring the driver, further comprises:
observing a behavior of the driver during the trip; and
comparing the behavior against baseline driving profile and a database of the medical event associated with one or more medical issues.

4. The computer-implemented method of claim 1, wherein generating the initial action list further comprises:
determining one or more solutions, through artificial intelligence, based on risk factors wherein the one or more solutions ensure the safety of the driver and surrounding vehicles and the risk factors comprises of weather, time of drive, traffic, speed of the vehicle, type of vehicle, status of the driver after the medical incident, and status of nearby first responders.

5. The computer-implemented method of claim 1, wherein the medical event further comprises, seizures, diabetes, epilepsy, intoxication and heart condition and driving impairment comprises of drunkenness and substance abuse.

6. The computer-implemented method of claim 1, wherein the initial action list further comprises, alerting the driver to be more attentive, activating internal and external cameras and alerting other nearby vehicles based on a medical condition of the driver.

7. The computer-implemented method of claim 1, wherein the subsequent action list further comprises, steering the vehicle to safety and/or to the nearest medical treatment facility, alerting medical personnel at the nearest facility, slowing down the vehicle and activating safety devices in the vehicle.

8. A computer program product for providing assistance to an operator of a vehicle during a medical event and/or driving impairment, the computer program product comprising:
one or more computer readable storage media and program instructions stored on the one or more computer readable storage media, the program instructions comprising:
program instructions to determine driving profile of a driver;
program instructions to monitors the driver during a trip;
program instructions to identifies an occurrence of the medical event and/or and driving impairment associated with the driver;
in responsive the occurrence of the medical event, program instructions to generate an initial action list;
program instructions to execute the initial action list;
program instructions to determine whether the driver is cognizant;
in responsive to the driver is not cognizant, program instructions to generate a subsequent action list; and
program instructions to execute the subsequent action list.

9. The computer program product of claim 8, wherein program instructions to determine driving profile of the driver, further comprises:
program instructions to generate a baseline driving profile based on a predetermined period of time associated with an observation of the driver; and
program instructions to store the baseline driving profile.

10. The computer program product of claim 9, wherein program instructions to monitor the driver, further comprises:
program instructions to observe a behavior of the driver during the trip; and
program instructions to compare the behavior against baseline driving profile and a database of the medical event associated with one or more medical issues.

11. The computer program product of claim 8, wherein program instructions to generate the initial action list further comprises:
program instructions to determine one or more solutions, through artificial intelligence, based on risk factors wherein the one or more solutions ensure the safety of the driver and surrounding vehicles and the risk factors comprises of weather, time of drive, traffic, speed of the vehicle, type of vehicle, status of the driver after the medical incident, and status of nearby first responders.

12. The computer program product of claim 8, wherein the medical event further comprises, seizures, diabetes, epilepsy, intoxication and heart condition and driving impairment comprises of drunkenness and substance abuse.

13. The computer program product of claim 8, wherein the initial action list further comprises, alerting the driver to be more attentive, activating internal and external cameras and alerting other nearby vehicles based on a medical condition of the driver.

14. The computer program product of claim 8, wherein the subsequent action list further comprises, steering the vehicle to safety and/or to the nearest medical treatment facility, alerting medical personnel at the nearest facility, slowing down the vehicle and activating safety devices in the vehicle.

15. A computer system for providing assistance to an operator of a vehicle during a medical event and/or driving impairment, the computer system comprising:
one or more computer processors;
one or more computer readable storage media; and
program instructions stored on the one or more computer readable storage media for execution by at least one of the one or more computer processors, the program instructions comprising:
program instructions to determine driving profile of a driver;
program instructions to monitors the driver during a trip;
program instructions to identifies an occurrence of the medical event and/or and driving impairment associated with the driver;
in responsive the occurrence of the medical event, program instructions to generate an initial action list;
program instructions to execute the initial action list;
program instructions to determine whether the driver is cognizant;
in responsive to the driver is not cognizant, program instructions to generate a subsequent action list; and
program instructions to execute the subsequent action list.

16. The computer system of claim 15, wherein program instructions to determine driving profile of the driver, further comprises:
program instructions to generate a baseline driving profile based on a predetermined period of time associated with an observation of the driver; and
program instructions to store the baseline driving profile.

17. The computer system of claim 15, wherein program instructions to monitor the driver, further comprises:
program instructions to observe a behavior of the driver during the trip; and
program instructions to compare the behavior against baseline driving profile and a database of the medical event associated with one or more medical issues.

18. The computer system of claim 15, wherein program instructions to generate the initial action list further comprises:
program instructions to determine one or more solutions, through artificial intelligence, based on risk factors wherein the one or more solutions ensure the safety of the driver and surrounding vehicles and the risk factors comprises of weather, time of drive, traffic, speed of the vehicle, type of vehicle, status of the driver after the medical incident, and status of nearby first responders.

19. The computer system of claim 15, wherein the medical event further comprises, seizures, diabetes, epilepsy, intoxication and heart condition and driving impairment comprises of drunkenness and substance abuse.

20. The computer system of claim 15, wherein the initial action list further comprises, alerting the driver to be more attentive, activating internal and external cameras and alerting other nearby vehicles based on a medical condition of the driver.
